(19) United States
(12) Patent Application Publication
(10) Pub. No.: US 2004/0180018 A1
(43) Pub. Date: Sep. 16, 2004

(54) Method and apparatus for hair care

(76) Inventor: Adrian O. Scott, DeKalb, TX (US)

Correspondence Address: Wood & Eisenberg, 2009 North 14th Street, Suite 706, Arlington, VA, 22201, US

(21) Appl. No.: 10/383,534

(22) Filed: Mar. 10, 2003

(51) Int. Cl. A61K 7/06; A61K 7/11

(52) U.S. Cl. 424/70.11; 442/123

(57) ABSTRACT

The method for hair care includes the steps of washing hair, rinsing the hair, drying the hair with a polyvinyl alcohol (PVA) towel, and adding conditioner to the hair. The use of a PVA towel to dry the hair reduces the amount of a hair conditioner required to condition a person's hair, such as a customer's hair in a hair salon. The apparatus for hair care is a hair treatment kit which contains at least a container of hair conditioner and a polyvinyl alcohol towel, and may optionally include a container of shampoo.

### BACKGROUND OF THE INVENTION

1. Field of the Invention

The present invention relates to an improved method and apparatus for hair care. More specifically, the invention is directed to a method and apparatus of hair care wherein the amount of hair conditioner required to adequately condition a person's hair is substantially reduced.

2. Description of the Related Art

A person's hair comprises a cuticle and a cortex. The cuticle is the outer layer of the hair which surrounds the cortex. The cuticle is made of pliable, scale-like cells. The cortex defines an inner region which can fill with water and/or conditioner. When the cortex is filled with conditioner the hair is said to have “body”. Since conditioner tends not to evaporate like water, the hair can maintain body when the person's hair is substantially dry.

Hair is usually washed by following a number of definite steps. For example, the hair is made initially wet with water (wetting step), shampoo (or its equivalent) is then added to the hair and rubbed or massaged into the hair to clean (washing step), the hair is then rinsed with water (rinsing step), and conditioner added to the hair (conditioning step). If the conditioner does not fill the cortex, then it tends to stick to the outer cuticle where it may act like a magnet for dust and dirt that adheres to the conditioner and hence to the cuticles of the hair.

When hair is contacted with water, the water tends to fill the cortex. Thus, when conditioner is added to the water-soaked hair, the conditioner is immediately in competition with the water to fill the cortex of the hair. Ideally the water should be removed from the cortex and replaced with conditioner, but quite often a lot of the water remains and results in hair filled with dilute conditioner (i.e., water and conditioner).

Water can be removed from the wet hair prior to adding the hair conditioner by squeezing the hair to force the water out of the cortex or rubbing the hair with a dry towel for a sufficiently long time in order to draw the water out of the hair by capillary action. Alternatively, the hair can be saturated with excess conditioner, or repeated applications of excess conditioner can be made, but this is expensive and time consuming and places an unwelcome burden on a professional hair salon. None of these techniques are satisfactory. Thus, there is a need for a new way of removing water from the hair.

Several efforts have been made to address these problems. U.S. Pat. No. 5,620,786, issued Apr. 15, 1997 to Honeycutt et al., describes towel, sponge and gauze products composed of fibers of polyvinyl alcohol resin produced by a process of dope extrusion. The '786 patent does not teach or suggest the use of, for example, a towel or like item for extracting water from the cortex of a person's hair in order to reduce the amount of hair conditioner required to condition the hair.

U.S. Pat. No. 5,304,420, issued Apr. 19, 1994 to Hirakawa et al., describes fibers and yarns composed of a vinyl alcohol unit-containing polymer having high water absorption unaffected by dyeing treatment or cleaning. The '420 patent does not teach or suggest a regime of hair care wherein a towel made of polyvinyl alcohol (PVA) or the like is used to extract water from the cortex of a person's hair in order to reduce the amount of hair conditioner required to condition the hair.

PVA based towels are well known and include the “PVA MAGIC TOWEL” (supplied by HANDY-AGE INDUSTRIAL CO., LTD., http://handage.com.tw/house/1-4.html) and the “The Sammy Sport Towel” (supplied by Shima American Corp., http://www.thesammy.com). However, neither the “PVA MAGIC TOWEL” nor “The Sammy Sport Towel” teach or suggest a regime of hair care wherein a towel made from PVA, or the like, is used to extract water from a person's hair in order to reduce the amount of hair conditioner required to condition the hair.

None of the above inventions and patents, taken either singly or in combination, is seen to describe the instant invention as claimed. Thus an improved method of hair care solving the aforementioned problems is desired.

### SUMMARY OF THE INVENTION

The present invention is a method and apparatus for hair care. The method for hair care of the present invention includes the steps of washing hair, rinsing the hair, drying the hair with a polyvinyl alcohol (PVA) towel, and adding conditioner to the hair. The use of a PVA towel to dry the hair reduces the amount of a hair conditioner required to condition a person's hair, such as a customer's hair in a hair salon. The apparatus for hair care of the present invention is a hair treatment kit which contains at least a container of hair conditioner and a polyvinyl alcohol towel. The hair treatment kit is especially useful for hair salons, and can be marketed with towels manufactured to have sufficient water absorption capacity to dry hair of a specified length (e.g., short, medium, long, etc.) within a specified period of time in order to increase efficiency and productivity in the hair salon.

Accordingly, it is an object of the invention to provide a method of conditioning hair which involves drying the hair with a towel made with polyvinyl alcohol prior to applying conditioner for improving the hair's capacity for receiving conditioner in the cortex of the hair, resulting in better hair body.

It is a further object of the invention to provide a method of hair care which includes drying the hair with a PVA towel before applying conditioner to the hair in order to reduce the amount of time required for conditioning the hair, either in a commercial hair care facility or in an ordinary home setting.

It is a further object of the invention to provide a range of towels comprising of PVA with specific ranges of water drawing power to optimize the turn around time of hair salon customers with wet hair who require a conditioner added to their hair.

It is another object of the invention to provide a kit adapted for use in a hair care salon to allow a salon operator to optimize the turn-round of customers with wet hair who require a conditioner added to their hair.

It is an object of the invention to provide improved elements and arrangements thereof for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is an environmental perspective view of a hair stylist drying a client's hair according to the method of the present invention.

FIG. 2 is a perspective view of a hair conditioning kit, with a polyvinylalcohol (PVA) towel, according to the invention.

FIG. 3A shows the hair conditioning kit with packaging and a hair conditioning kit contained therein, and words printed on the packaging according to the present invention.

FIG. 3B shows exemplary words printed on the packaging shown in FIG. 3A.

FIG. 4A shows a label with words printed thereon according to the present invention.

FIG. 4B shows a further label with words printed thereon according to the present invention.

Similar reference characters denote corresponding features consistently throughout the attached drawings.

### DETAILED DESCRIPTION OF THE PREFERRED EMBODIMENTS

The present invention relates to an improved method and apparatus for hair care. More specifically, the invention is directed to a method and apparatus of hair care wherein the amount of hair conditioner required to adequately condition a person's hair is substantially reduced.

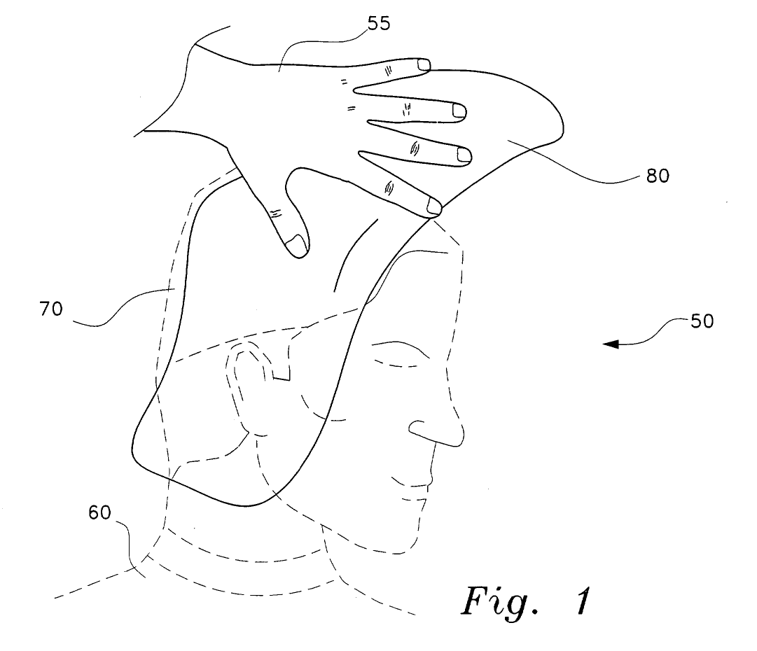

FIG. 1 is an environmental perspective view 50 of a hair stylist 55 working on a client 60, and more particularly the client's wet hair 70, according to the method of the present invention. The hair stylist 55 is shown drying, by means of a rubbing action, the client's wet hair 70 using a polyvinylalcohol towel 80 according to the invention.

The terms “polyvinylalcohol” and “PVA” are hereinafter regarded as equivalent terms. The terms “customer”, and “client” are hereinafter regarded as equivalent terms. However, it should be understood that the method and apparatus described herein is also directed to non-saloon use by e.g. persons who wash and take care of their hair in an ordinary home setting. Non-limiting examples of other persons' hair that the present invention is directed include, but is not limited to, the conditioning of hair belonging to: parents of both sexes, children of both sexes, students (of both sexes) resident in student dorms, single adults (of both sexes), military personnel (of both sexes), and non-military personnel (of both sexes). Thus, the present invention is expressly not limited to use in professional hair saloons but applies equally to the treatment and care of hair in other places such as in ordinary homes and residences and any place where people wash and condition their own hair.

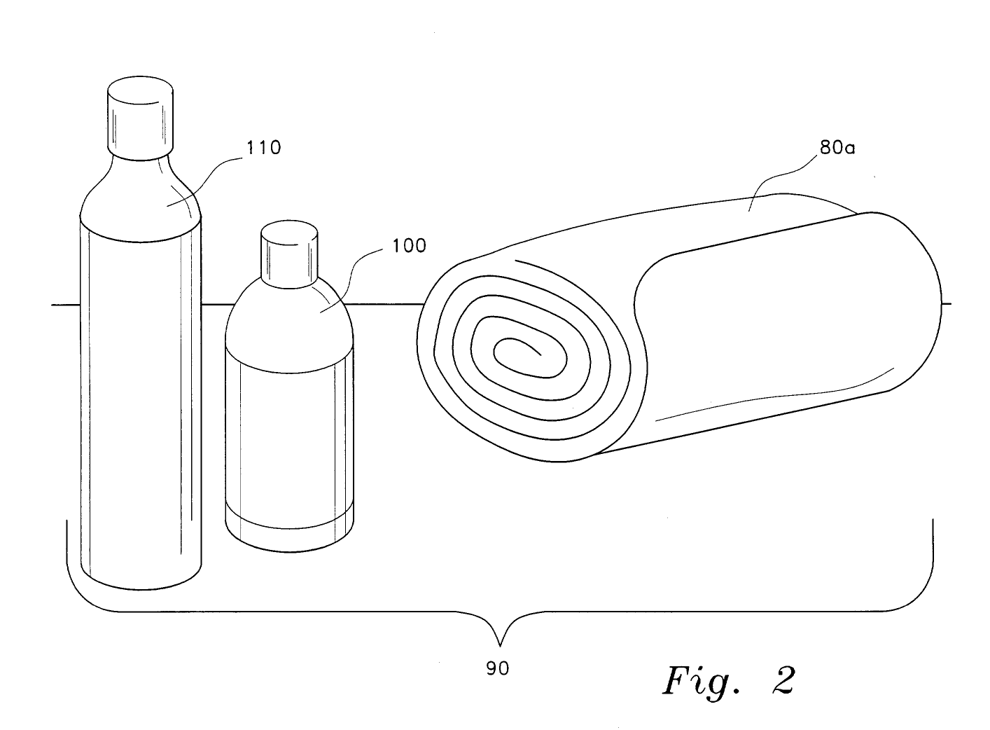

FIG. 2 is a perspective view of a hair conditioning kit 90 according to the invention. The hair conditioning kit 90 has at least one each of: a polyvinylalcohol towel 80a, a container of conditioner 100, and a container of shampoo 110. The container of shampoo 110 is optional, though it is preferred that the kit 90 includes the shampoo container 110.

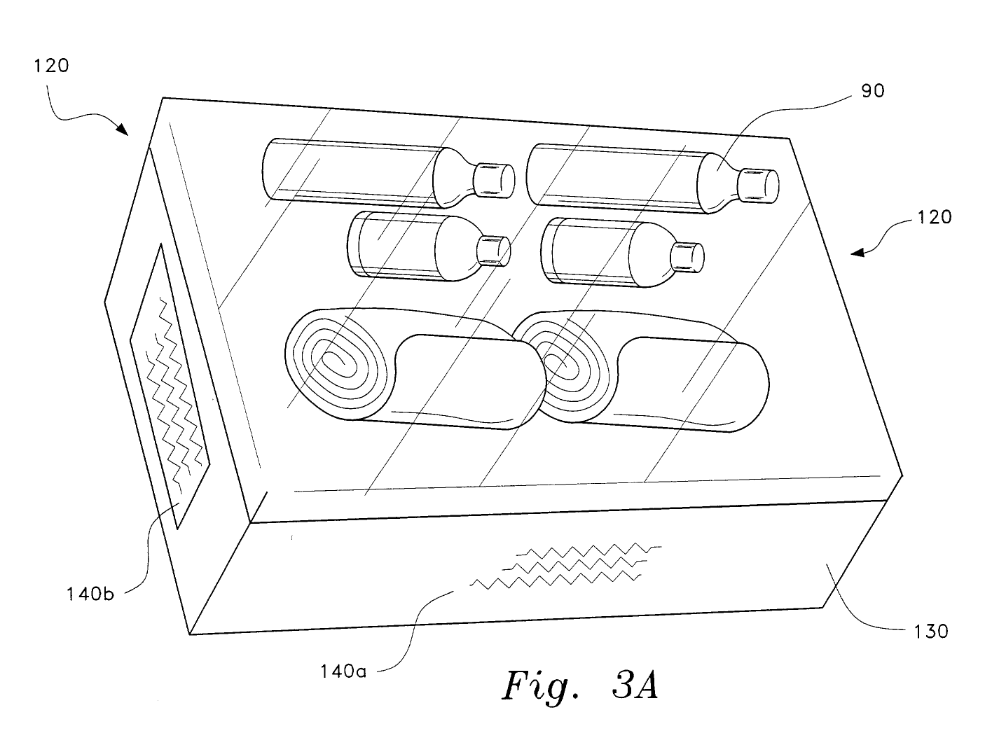

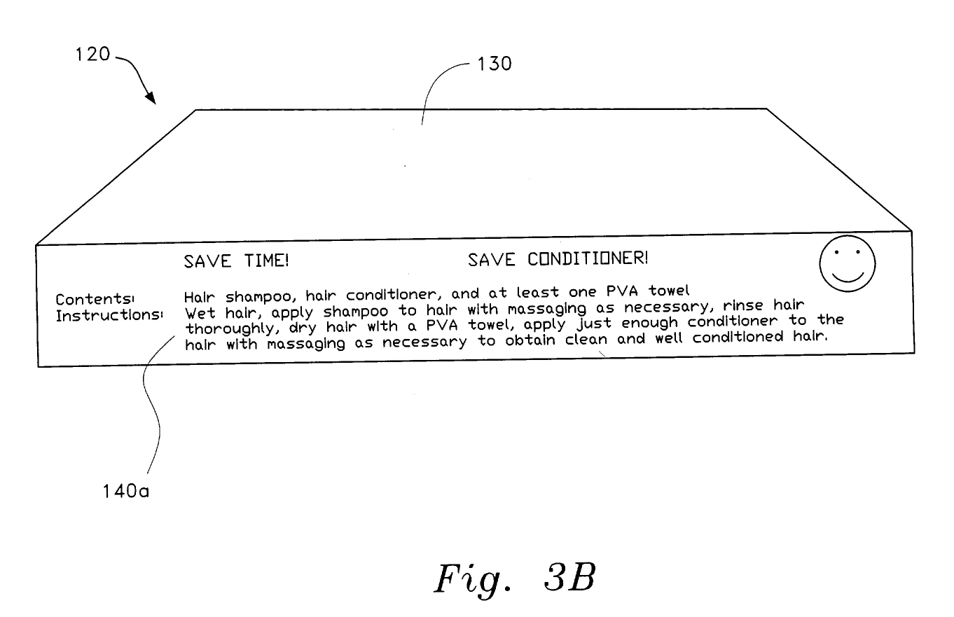

Referring to FIGS. 3A and 3B, FIG. 3A shows an article of manufacture 120 with packaging 130 including the hair conditioning kit 90 housed therein, and words 140a printed on the packaging 130 according to the present invention. The words 140a (see FIG. 3B) describe the contents of the package 130 (i.e. the hair conditioning kit 90) and instructions for using the kit 90 for cleaning and conditioning a person's hair. The indicia 140a include an indication that the kit 90 requires a reduced amount of conditioner for conditioning hair (“SAVE CONDITIONER”).

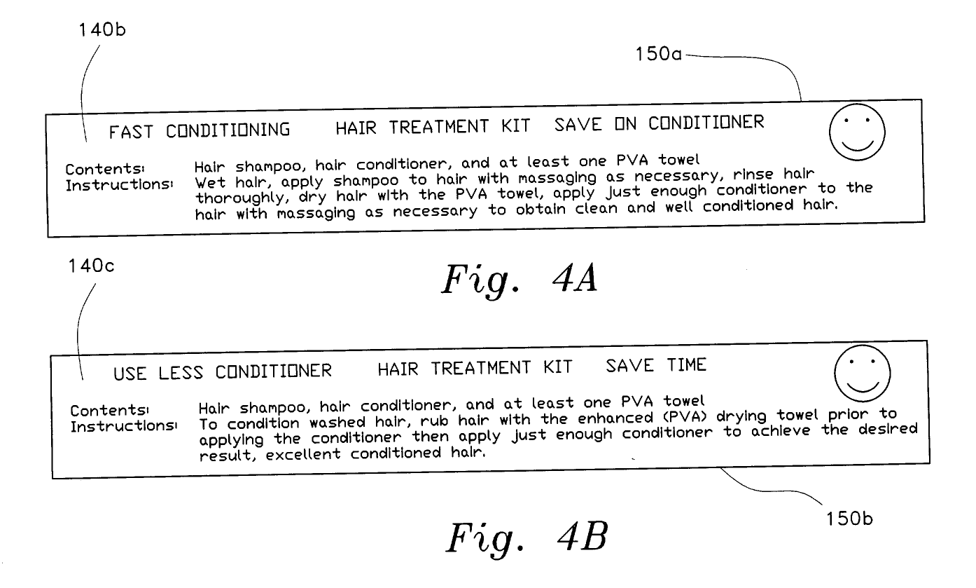

Referring to FIGS. 4A and 4B, FIG. 4A shows a label 150a with words 140b printed thereon that indicate that the article of manufacture 120 can be used to condition a person's hair while reducing the amount of conditioner required to condition a person's hair. FIG. 4B shows a label 150b with alternative words 140c printed thereon that indicate that the article of manufacture 120 can be used to condition a person's hair while reducing the amount of conditioner and time required to condition a person's hair according to the present invention. The labels 150a and 150b can have a sticky reverse side that enable the labels 150a and/or 150b to be affixed to the article of manufacture 120 in place of, or in addition to, the words 140a printed on the surface of the packaging 130 (see FIGS. 3A and 3B).

In a further embodiment of the invention an improved method of hair care is provided in which the amount of a hair conditioner 100 required to condition a person's hair 70 is significantly reduced. The method comprises the steps of: washing a persons hair 70, preferably using a hair shampoo 110; rinsing the person's hair 70; drying the person's hair 70 with a towel made with polyvinyl alcohol 80 (by rubbing the person's hair 70 with the towel 80 or by pressing the person's hair 70 with the towel 80 or by a combination of rubbing and pressing the person's hair 70 with the towel 80); and adding a sufficient amount of a hair conditioner 100 to condition the person's hair 70.

The step of drying the person's hair 70 with the towel made with polyvinyl alcohol significantly reduces the amount of hair conditioner otherwise required to condition the person's hair 70 if a polyvinyl alcohol towel 80 was not used, i.e. the amount of conditioner required is reduced by at least 50′ (see Table 1 for the kind of results that would be expected in a commercial operation). In a commercial operation, such as a retail chain of hair salons, a reduction in requirement of conditioner of 50% or more is considered herein as a significant or substantial reduction. This significant reduction is achieved by the step of rubbing a polyvinyl alcohol towel 80 on a person's wet hair 70 for about ten seconds.

While the size of the polyvinyl alcohol towel 80 can vary the preferred size from a handling perspective is about 25 cm by 17 cm by 2 cm. It is preferred that the polyvinyl alcohol towel 80 contains sufficient polyvinyl alcohol in order to absorb from about 2 fluid ounces to about 15 fluid ounces of of water from wet hair in about five to twenty seconds of rubbing the person's hair with the polyvinylalcohol towel 80; more preferably, the towel 80 contains sufficient polyvinyl alcohol in order to absorb from about 3 fluid ounces to about 10 fluid ounces of water from wet hair in about five to twenty seconds of rubbing the person's hair with the polyvinylalcohol towel 80; and still more preferably, the towel contains sufficient polyvinyl alcohol in order to absorb from about three fluid ounces to about five fluid ounces of water from wet hair in about ten seconds of rubbing the person's hair with the polyvinylalcohol towel 80.

By using a polyvinylalcohol towel 80 to remove water from a person's wet hair, the amount of conditioner used to condition the person's hair is reduced by about two-thirds compared to not rubbing a person's wet hair with a polyvinyl alcohol towel or rubbing a person's hair with an ordinary towel with no polyvinyl alcohol component. The amount of water absorbed may be estimated by rubbing a person's hair with a dry polyvinyl alcohol towel 80 for about ten seconds and then wringing out the polyvinylalcohol towel and measuring the wrung out water in a fluid ounce measuring cup. Table 1 summarizes the results of tests made according to this testing method. The amount of hair conditioner saved is at least about 50% of the amount of conditioner that would otherwise be used absent using a dry PVA towel to rub a customer's hair 70.

For example, about a ⅔rd saving on conditioner was achieved with respect to customers #2 and #3 (see Table 1). With respect to customers #2, #3, and #5 where the amount of conditioner saved was recorded (see column 4 of Table 1) the amount of conditioner saved was substantial and significant, i.e. at least 50% and as much as a ⅔rd saving with respect to the normal amount of conditioner used for the hair type (see column 2 of Table 1). Thus, it should be understood that the terms “substantial reduction” or “significant reduction” refers to a saving of at least 50% and as much as ⅔rds (i.e. 66.6%). Such a substantial and significant saving of conditioner represents a considerable saving in cost with respect to, for example, the operating costs of a commercial hair salon. In each of the studied cases (#1 through to #5, column 1, Table 1) the ability of the hair to stretch (by applying tension to the hair) and return to the pre-stressed length was checked, and in each case the hair returned to its previous pre-stress length indicating good hair resilience. A curl test was also performed wherein a hair curl was stressed and checked to see if the tested curl returned to its original state. This test also produced good results on each of the five cases. It should be clearly understood that the data presented in Table 1 is not intended to limit the scope of the present invention in any way; specifically, the present invention is directed to the conditioning of hair of any person and is not limited to treating the hair of salon customers, but applies equally to other peoples' hair, including (but not limited to) the conditioning of hair belonging to e.g., parents of both sexes, children of both sexes, students (of both sexes), single adults (of both sexes), military personnel (of both sexes), and non-military personnel (of both sexes).

It is to be understood that the present invention is not limited to the embodiments described above, but encompasses any and all embodiments within the scope of the following claims.

TABLE 1

| | | | Amount of |
|---|---|---|---|
| | | Amount of | conditioner used in |
| | | water absorbed | grams (gm) on the |
| | | (fluid oz) by the | client (amount in |
| Customer | | PVA towel* in | parenthesis indicates |
| # | Hair type | about 10 seconds** | usual amount used) |
| 1 | long hair (13″); | 4.5 | 5.6 |
| | medium texture, | | |
| | normal density | | |
| 2 | 10″ length hair | 4.0 | 4.2 (14.0) |
| 3 | 4″ (about bob | 3.0 | 3.3 (10.0) |
| | length) | | |
| 4 | short hair (3″), | 2.5 | 3.0 |
| | coarse hair | | |
| 5 | 2″ short hair | 2.0 | 2.4 (7.0) |

*PVA towel with dimensions 26.9 cm by 16.9 cm by 2 cms thick reinforced with nylon mesh supplied by Perfect & Glory of Twain (7-2f, 431, Kuang Fu S. Rd., Taipei, Taiwan); described as a three layered heat cured and laminated PVA towel.
**about 10 seconds of rubbing a dry PVA towel in the customer's hair.
***Gram measurements may be performed using a digital scale and zeroing the scale on a weighing dish prior to taking each gram reading.

I claim:

1. A method of hair care in which the amount of a hair conditioner required to condition a person's hair is significantly reduced, said method comprising the steps of:
washing a persons hair;
rinsing the person's hair;
drying the person's hair with a polyvinyl alcohol towel; and
treating the person's hair with hair conditioner;
whereby the amount of hair conditioner required to condition the person's hair is reduced by efficient drying of the person's hair.

2. The method of hair care of claim 1, wherein the step of drying the person's hair with the polyvinyl alcohol towel comprises rubbing the person's hair with the polyvinylalcohol towel.

3. The method of hair care of claim 1, wherein the towel comprises sufficient polyvinyl alcohol in order to absorb from about two fluid ounces to about fifteen fluid ounces of water from wet hair in about five to twenty seconds of contacting the person's hair with the towel.

4. The method of hair care of claim 1, wherein the towel comprises sufficient polyvinyl alcohol in order to absorb from about three fluid ounces to about ten fluid ounces of water from wet hair in about five to twenty seconds of contacting the person's hair with the towel.

5. The method of hair care of claim 1, wherein the towel comprises sufficient polyvinyl alcohol in order to absorb from about three fluid ounces to about five fluid ounces of water from wet hair in about five to twenty seconds of contacting the person's hair with the towel.

6. The method of hair care of claim 1, wherein the towel comprises sufficient polyvinyl alcohol in order to absorb from about three fluid ounces to about five fluid ounces of water from wet hair in about ten seconds of contacting the person's hair with the towel.

7. A hair treatment kit for reducing the amount of hair conditioner required to condition a person's hair, comprising:
at least one container having hair conditioner contained therein; and
at least one polyvinyl alcohol towel capable of rapidly absorbing water from a person's wet hair;
whereby the amount of conditioner required to condition the person's hair is reduced.

8. The hair treatment kit of claim 7, wherein the polyvinyl alcohol towel comprises sufficient polyvinyl alcohol in order to absorb from about two fluid ounces to about fifteen fluid ounces within about twenty seconds of applying the polyvinyl alcohol towel to the person's wet hair.

9. The hair treatment kit of claim 7, wherein the polyvinyl alcohol towel comprises sufficient polyvinyl alcohol in order to absorb from about three fluid ounces to about ten fluid ounces within about twenty seconds of applying the polyvinyl alcohol towel to the person's wet hair.

10. The hair treatment kit of claim 7, wherein the polyvinyl alcohol towel comprises sufficient polyvinyl alcohol in order to absorb from about three fluid ounces to about five fluid ounces within about ten seconds of applying the polyvinyl alcohol towel to the person's wet hair.

11. An article of manufacture for reducing the amount of hair conditioner required to condition a person's hair, comprising:
at least one polyvinyl alcohol towel;
at least one container having hair conditioner contained therein; and
a package, the towel and the container being disposed within the package;
wherein the package has information printed thereon; and
wherein the printed information indicates that the article of manufacture can be used for conditioning a person's hair with less than the usual amount of a hair conditioning agent.

12. The article of manufacture of claim 11, further comprising at least one container having shampoo contained therein.

13. The article of manufacture of claim 11, further comprising at least one container having shampoo contained therein, and wherein the printed information further indicates that the article of manufacture can be used to condition a person's hair while reducing the amount of conditioner and time required to condition a person's hair.

14. The hair treatment kit of claim 11, wherein the information is printed on a label affixed to the packaging.
